(19) United States
(12) Patent Application Publication
(10) Pub. No.: US 2026/0083316 A1
(43) Pub. Date: Mar. 26, 2026

(54) WHITE-LIGHT SOURCE AND INTRAOCULAR ILLUMINATION DEVICE

(71) Applicant: CARL ZEISS MEDITEC AG, Jena (DE)

(72) Inventors: Ralf DOUBEK, Langenau (DE); Michelangelo MASINI, Aalen (DE); Friederike NEIS, Ulm (DE)

(21) Appl. No.: 19/109,457

(22) PCT Filed: Apr. 28, 2023
(86) PCT No.: PCT/EP2023/061319

§ 371 Date: Mar. 6, 2025

(30) Foreign Application Priority Data
May 23, 2022 (DE) 10 2022 112 922.5

(51) Int. Cl. A61B 3/00; A61B 90/30

(52) U.S. Cl. CPC A61B 3/0008; A61B 90/30; A61B 2090/306

(57) ABSTRACT

A white-light source for fiber-based intraocular illumination is light of controllable, spectrally broad composition and to an intraocular illumination device with corresponding controllability. Specifically, the white-light source for fiber-based intraocular illumination with light of controllable spectral composition includes at least two light sources for the provision of light beams of different colors, preferably with components substantially in the blue, the green and the red spectral range; wherein the individual light beams are combined to form a common light beam (W); wherein the white-light source is configured for individual control of the proportions of the individual light beams in the common light beam (W); wherein at least one of the light sources is a laser-activated remote phosphor light source, LARP light source, having a phosphor as conversion element and a laser diode for exciting the conversion element by means of an excitation radiation (S) emitted by the laser diode.

### DESCRIPTION

The present invention relates to a white-light source and an intraocular illumination device comprising the white-light source. In particular, the invention relates to a white-light source for fiber-based intraocular illumination with light of controllable spectrally broad composition and to an intraocular illumination device with corresponding controllability.

### PRIOR ART

For intraocular illumination, for example during posterior segment surgery, light is supplied to the eye with the aid of light guides such as optical fibers or other fibers. In this context and within the scope of the invention, the term “fiber” is used synonymously with “light guide”. These fibers have a diameter that is as small as possible and are introduced into the interior of an eye on which surgery should be performed. To this end, the fiber is introduced into the vitreous humor by means of a small incision at the edge of the eye. In this context, the distal end of the fiber may take different forms (e.g. a lensed fiber) depending on the demands placed on the illumination in each case, and so the posterior segment of the eye may be illuminated either at a point or broadly, in accordance with the respective surgical requirements. Since the incision on the eye should be kept as small as possible, there is an upward limit on the admissible fiber diameters. Then again, luminous fluxes that are as high as possible should be able to be introduced into the eye through the fiber so as to ensure an optimal illumination of the surgical area. Therefore, the essential object of intraocular illumination devices is that of being capable of supplying very large amounts of light to the eye using fiber diameters that are as small as possible.

A further object of such devices is that of being capable of providing a good adaptation of the spectral composition of the light to the respective application. In particular, this relates to the ability of tuning the spectral composition (setting the color temperature) of frequently required white light. Corresponding light sources for the provision of light with a controllable, spectrally broad composition substantially in the visible spectral range (VIS spectral range) are referred to as spectrally controllable white-light sources. Within the scope of the invention, the terms “controllable” and “controllability” should in particular be understood to refer to both open-loop and closed-loop control. Moreover, the light should however also be able to provide good color rendering. To this end, the illumination device should be able to provide a color space that is as large as possible and that has a spectrum in the visible spectral range which is covered as continuously as possible. An ophthalmologist often makes their decisions on the basis of only fine color shades. These color shades can only be observed and evaluated accordingly should the light used for illumination purposes comprise corresponding wavelengths. In particular, a natural reproduction close to the solar light spectrum (or how this would look through the respective surgical microscope) is preferred. Where possible, a medical decision in this context should not be based on different illumination situations.

To date, lamps based on xenon or halogens were predominantly used as light sources. In an alternative to that, light-emitting diodes (LEDs) are also used. In this case, what are known as white-light LEDs, in which white light is usually created by a phosphor that emits spectrally broadly in the event of excitation with blue light, and additive white-light sources, in which the light emitted by red, green and blue LEDs is mixed to form white light, in particular are used for illumination devices. In both cases, the amount of light that can be input coupled into the optical fiber is limited inter alia by a correspondingly low étendue of the emitted light. The proximal fiber end and its surroundings may melt in the event of a power density that is too high and may be destroyed as a result. Moreover, should phosphor-based white-light LEDs be used, it is moreover not possible to control the color temperature of the emitted light on account of the predetermined conversion properties of the utilized phosphor, and so the emitted light must initially be split into individual spectral components (e.g. by way of appropriate spectral filter elements) and subsequently be recombined again in accordance with the respective spectral requirements.

The use of laser diodes (LDs) in place of LEDs is also possible. In this case, too, the light emitted by red, green and blue LDs can be mixed to form white light. However, such white light only enables significantly restricted color rendering on account of its spectral bandwidth, which is significantly reduced vis-à-vis corresponding LEDs. However, the advantage of using LDs as light sources lies in their very high étendue, which allows efficient input coupling of the emitted light even into very small fiber diameters. A further disadvantage lies in the strong coherence of the emitted radiation, whereby bothersome interference effects may form, and it may be necessary to take account of laser protection standards. Additional diffusers or other elements that reduce the coherence of the radiation are able to reduce this but lead to further losses and reduce the étendue.

In the meantime, LARP white-light sources (laser-activated remote phosphor light sources) have been developed; in these, the conversion principle known from LEDs was transferred to LDs. In this case, the radiation emitted by the LD is focused at a phosphor as conversion element, converted into a corresponding white-light spectrum there and collected again by the associated focusing optics unit. On account of the punctiform excitation, the high étendue of the laser radiation is largely maintained during the conversion process, but the strong coherence of the excitation radiation is largely lost during the conversion process. In comparison with the use of LEDs, such LARP white-light sources allow a significantly higher light intensity to be input coupled into fibers with diameters that are even significantly smaller than 0.5 mm. In this respect, the known LARP white-light sources create a substantially coherence-free light with high étendue. However, LARP white-light sources have not been used for intraocular illumination purposes to date.

### DISCLOSURE OF THE INVENTION

Therefore, a problem addressed by the present invention is that of specifying a white-light source and an intraocular illumination device which are able to provide light with a particularly high étendue, low coherence and controllable, spectrally broad composition for fiber-based intraocular illumination.

According to the invention, these problems are solved by the features of independent claims 1 and 12. Advantageous configurations of the invention are contained in the respective dependent claims.

One aspect of the present invention relates to a white-light source for fiber-based intraocular illumination with light of controllable spectral composition, comprising at least two light sources for the provision of light beams of different colors, preferably with components substantially in the blue, the green and the red spectral range; wherein the individual light beams are combined to form a common light beam; wherein the white-light source is configured for individual control of the proportions of the individual light beams in the common light beam; wherein at least one of the light sources is a laser-activated remote phosphor light source, LARP light source, having a phosphor as conversion element and a laser diode for exciting the conversion element by means of an excitation radiation emitted by the laser diode.

By preference, the white-light source comprises one light source each for the provision of single-color light beams with respective components substantially in the blue, the green and the red spectral range. By preference, the white-light source comprises a beam combiner for combining individual light beams to form the common light beam.

In particular, the present invention relates to a white-light source for fiber-based intraocular illumination with light of controllable spectral composition, comprising one light source each for the provision of single-color light beams with respective components substantially in the blue, the green and the red spectral range; and a beam combiner for combining the individual light beams to form a common light beam; wherein the white-light source is configured for individual control of the proportions of the individual light beams in the common light beam; wherein at least one of the light sources is a laser-activated remote phosphor light source, LARP light source, having a phosphor as conversion element and a laser diode for exciting the conversion element by means of an excitation radiation emitted by the laser diode.

In this case, a white-light source refers to a light source which, at least in principle, is suitable for the provision of white light with a spectral distribution that is as broad as possible in order to allow color rendering that is as complete as possible, and the emitted white-light spectrum of which thus arises as a mixture of red, green and blue spectral components. In this case, the spectral components may be characterized in particular as narrow-bandwidth-separated regions (e.g. if LDs of different colors are used) or by at least one continuous spectral band (e.g. if a white-light LED or white-light LARP is used). In this case, the adaptation of the white overall spectrum in respect of the intensities of the individual components or their spectral distribution is referred to as controllability of the spectral composition.

Thus, a white-light source according to the invention is an RGB light source, the RGB composition of which can be chosen as freely as possible. In particular, a white-light source according to the invention should therefore also be designed for the provision of two-color or single-color light from only two light sources or only a single light source by way of corresponding down-regulation and/or filtering of a light source or individual light sources from the comprised light sources. For example, in this case a single light source may also be designed for an emission of dichromatic light, i.e. light of different colors with components substantially in two spectral ranges from the group of the blue, the green and the red spectral range. However, by preference, a single light source for a combined provision of white light according to the invention only emits single-color light, i.e. light of in each case only a single color with components substantially in one spectral range from the group of the blue, the green and the red spectral range, on account of the better controllability.

The white-light source according to the invention comprises one light source each for the provision of single-color light beams with respective components substantially in the blue, the green and the red spectral range. In this context, the term “single color” relates to the respective associated spectral regions of the light source which in particular arise from the color impression to an observer. In this case, in particular, the wavelength ranges from approximately 640 nm to approximately 780 nm, from approximately 490 nm to approximately 570 nm and from approximately 430 nm to approximately 490 nm are generally referred to as red, green and blue, respectively. Corresponding transition regions (e.g., greenish blue, yellowy green, yellowy orange) are distinguished between these wavelength ranges. Since the human eye only has receptors for light from the aforementioned wavelength ranges, the entire range is also referred to as visible spectral range (VIS spectral range). The violet (in the wavelength range from approximately 380 nm to approximately 430 nm) and ultraviolet spectral ranges (UV spectral range, starting in the wavelength range below approximately 380 nm) adjoin below the blue spectral range. The infrared spectral range (IR spectral range, starting in the wavelength range above approximately 780 nm) adjoins above the red spectral range.

Hence, “single-color light” within the meaning of the present invention is defined by way of the color impression and may encompass a relatively broad spectrum (from the respective assigned spectral range) and thus even extend beyond the boundaries of the individual spectral ranges. Thus, single-color light within the scope of the invention may also be understood to mean light in the reddish yellow or bluish green or violety blue spectral range, for example. By contrast, the term “monochromatic light” as is generally known in the art represents light whose spectrum comprises substantially only a single wavelength (or a very narrow range about a certain central wavelength). A laser diode with an emission wavelength at 680 nm emits substantially monochromatic light while, by contrast, a red light-emitting diode transmits spectrally relatively broad, single-color (but not monochromatic) red light with components substantially in the red spectral range.

Depending on the spectral width of a light source, it is possible-as described above that the individual light beams also contain secondary spectral components that are located at a distance from the actual emission spectrum and outside of the actual or immediately adjacent spectral ranges (e.g. excitation or primary light in the event of converting light sources); therefore, the respective components are also defined as located substantially in the blue, the green and the red spectral range. This case depends on the respective color impression of an observer in particular; however, there can also be delimitation by way of the comparative intensity ratios of the individual spectral ranges of the emitted light.

The color impression may be described quantitatively by what is known as the “dominant wavelength” of the corresponding light. The dominant wavelength of a light may be determined on the basis of the corresponding color space representation as the point of intersection of the straight line that is defined by the color point of the light source and the white point with the closest edge of the color gamut. Narrow-bandwidth or mono-frequency light sources tend to be located at the edge of the color gamut while more broadband light sources tend to be situated in the center of the color gamut. The dominant wavelength defines the wavelength at which the emitted light is perceived as dominant in the human eye. Single-color light beams with respective components for example substantially in the red spectral range may therefore be located in the vicinity of the red edge of the color gamut, with their dominant wavelength being located between approximately 640 nm and approximately 780 nm. A corresponding statement applies to the respective other light colors.

The beam combiner may be an optical component or apparatus for combining the individual light beams to form a common light beam. For example, such beam combiners may take the form of cube-shaped elements, in which the input beams are input coupled into the optical element via three different side faces and the common beam is output coupled at the associated fourth side face (“X-cube”). Corresponding beam combiners may also be provided completely on a fiber basis (as what are known as fiber combiners). DE 10 2005 054 184 B4 has disclosed further embodiments of beam combiners. Beam combiners are part of the prior art and therefore sufficiently well known to a person skilled in the art.

The white-light source according to the invention is configured for individual control of the proportions of the individual light beams in the common light beam. This means that, for example, the intensity of the individual light sources can be controlled on an individual basis, or the white-light source according to the invention allows a controllable attenuation of the individual light beams following their provision by the individual light sources. To this end, an electronic control may be provided for the purpose of individually controlling the components of the individual light sources in the common light beam. In the simplest case, control may directly relate to the light creation in the individual light sources, for example the open-loop control of an operating current serving the closed-loop control of the light intensity emitted by the individual light sources. However, the electronic control may also relate to optical control of the light intensity following the provision of the light beams. Hence, the individual control relates to all means and measures suitable for varying the components of the individual light sources and is not restricted to an individual type of intensity control.

According to the invention, at least one of the light sources is a LARP light source having a phosphor as conversion element and a laser diode for exciting the conversion element by means of an excitation radiation emitted by the laser diode. LARP light sources are part of the prior art and sufficiently well known to a person skilled in the art. Particularly preferably, all light sources of the white-light source according to the invention take the form of LARP light sources.

The advantage of LARP light sources lies in their large étendue in combination with high light intensities in the event of low coherence of the light. As a result, high powers may also be input coupled efficiently into small fiber diameters without arising coupling losses leading to an inadmissible increase in temperature and hence to the melting of the proximal end of the utilized fiber used for input coupling purposes. By using LARP light sources, at least the spectral component associated with these light sources may be input coupled into a corresponding fiber with increased efficiency vis-à-vis the light from other light sources. In the event of exclusive use of LARP light sources in a white-light source according to the invention, the maximum light intensity that can be input coupled into a corresponding fiber may be significantly increased vis-à-vis other white-light sources from the prior art.

Hence, it is preferable according to the invention to mix three LARP light sources with individual spectra in the colors of red, green and blue. In this context, the respective spectra tend to be narrowband in comparison with the overall spectrum of a typical white-light LED but are significantly more broadband in comparison with a mixed RGB laser arrangement. In particular, this can be used to generate very broad red, green and blue spectral components, in order to be able to mix and individually control these. The greater spectral bandwidths of LARP light sources moreover allow the creation of good to very good color rendering. By dividing the power components among three light sources, it is in particular also possible to have higher powers than in the event of using conventional white-light LEDs. In contrast to direct white-light emitters with a continuous broadband spectrum, a reduced overall spectrum may moreover be created in a targeted fashion by way of the individual provision of the color components, whereby the ratio of amount of light to bandwidth can be improved. The components of the individual light beams can be regulated or controlled to a specific color location by way of an electronic control. In particular, the individual disadvantages of previous technology may be complemented or compensated for by the advantages of the respective other technology. LARP technology removes disadvantages due to typical laser properties such as long coherence lengths, laser speckle, poor color rendering and an otherwise necessary classification into laser classes.

By preference, the conversion element comprises an auxiliary phosphor for adapting the conversion element to the excitation radiation of the laser diode. Depending on the utilized LD for exciting specific phosphors as conversion element, it may be advantageous to include what is known as an auxiliary phosphor in the phosphor composition or to use said auxiliary phosphor in the corresponding assembly. An excitation radiation from an exciting LD and possibly not optimally designed from a spectral point of view may moreover be adapted to the specific absorption properties of the utilized phosphor with the desired target spectrum. Hence, the excitation radiation may be adapted by way of the auxiliary phosphor. By preference, however, the conversion element may also comprise a plurality of phosphors for specifying a certain converted color spectrum. In particular, this may increase the bandwidth of the conversion light. The individual phosphors may be mixed with one another during the production, and so these are present in a manner distributed as equally as possible within the conversion element. For example, the spectral distribution of a LARP light source that emits in the red, green or blue spectral range may be specifically adapted to the required illumination needs by the phosphor mixture used in the conversion element.

By preference, the at least one LARP light source is designed to emit white light. An adaptation of the spectral bandwidth or spectral curve of the emitted white light for the provision of the associated single-color light beam may be implemented by way of a spectral filter element between the corresponding LARP light source and the beam combiner or as an element of the beam combiner. This means that the broad spectral distribution of the emitted light from a white-light LARP is reduced or modified by an additional spectral filter element. The spectral distribution downstream of the spectral filter element then for example emerges as product of the spectral distribution of the white-light LARP and the spectral curve of the pass range of the spectral filter element. Within the scope of the invention, it is preferable for the pass range to be provided multiple times, in particular twice. For standards-related and/or statutory reasons, for example, the scope of the invention may include, for eye protection reasons, the arrangement of a further filter in the common light beam or else in the blue light beam, the proportion of the blue component incident on the eye being able to be reduced further using said further filter. This allows the duration of a surgical procedure to be increased.

The spectrum of a white-light LARP is typically very broad and allows very good color rendering even post spectral filtering. However, a disadvantage of this arrangement is that, as a result of filtering, only a fraction of the original light intensity is available for input coupling into an optical fiber since a portion of the white light is filtered out at the spectral filter element. The spectral filter element may also be integrated directly into the housing of a LARP light source (single-color LARP light source). The spectral filter element may also take the form of an element of the beam combiner, for example a dielectric filter layer applied directly to the surface of the beam combiner or to an internal interface of the beam combiner. By preference, the beam combiner performs such spectral mixing that no losses occur where possible, and there is a summation of the input powers.

By preference, the at least one LARP light source is directly designed for the provision of the associated colored light beam. By preference, there is a direct provision of the associated colored light beam by an adapted combination of phosphor and laser diode within the at least one LARP light source. In contrast to the embodiment with an additional spectral filter element, described in the preceding paragraph, a LARP light source that already emits a single color is used in this case. In particular, this may be achieved by virtue of the phosphor of the conversion element converting a corresponding excitation radiation into only a very specific spectral range. To this end, a plurality of heat-resistant phosphors for the different spectral ranges are available. The spectral distribution from a LARP light source designed directly for the provision of the associated colored light beam may however still be additionally adapted by a spectral filter element. In this case, the intensity loss at the spectral filter element may be reduced significantly vis-à-vis a monochromatically filtered white-light LARP.

By preference, the provision of the colored light beam with components substantially in the blue spectral range is implemented by way of a diode, LED, or laser diode, LD, emitting light substantially in the blue or violet spectral range. The provision of the colored light beams with components substantially in the red and the green spectral range is preferably implemented by means of one LARP light source each. This configuration is preferred since the blue or violet spectral range is only required to a small extent for color mixing and can hence be realized for example directly by way of an LED. The construction costs for a white-light source according to the invention may be reduced in this way in particular, since LEDs and LDs are significantly cheaper than corresponding LARP light sources, and no substantial disadvantages arise by the replacement of said LARP light sources in the blue spectral range.

By preference, the provision of the colored light beam with components substantially in the blue spectral range is implemented by way of a laser diode emitting light substantially in the blue or violet spectral range. Additionally, a conversion element for the provision of a single-color light beam with components substantially in the cyan spectral range may be implemented by way of the laser diode (LARP light source in the cyan spectral range).

The use of single-color light in the cyan spectral range for example enables direct driving of the fluorescein fluorescence in the eye and/or can be used to obtain a better color rendering value. The cyan spectral range is not defined uniformly in the prior art but is generally approximately assigned to the wavelength range from 482 nm to 494 nm, in particular between 487 nm and 492 nm. Thus, spectrally, the cyan spectral range is at the low-energy edge of the blue spectral range or very close to the green spectral range.

Since light in the blue spectral range has a higher energy than light in the green spectral range, the cyan spectral range can be provided in particular by conversion from blue or violet light by means of phosphors (“cyan phosphor”). Since—as already specified above—the blue spectral range is usually only required to a small extent for color mixing, a LARP light source for the provision of a single-color light beam with components substantially in the cyan spectral range can therefore be realized using a component of the blue laser radiation created. The light beams with components substantially in the blue spectral range and in the cyan spectral range may in this case have a common light path in the direction of the beam combiner. The white-light source according to the invention may therefore be constituted in such a way that, in particular, a greater amount of blue or violet light than required for a certain predetermined correlated color temperature (CCT) is initially created internally. This amount can then be converted by means of a cyan phosphor, in order to obtain a white light or filtered colored light with exactly specified properties.

By preference, the white-light source according to the invention furthermore comprises a LARP light source as light source for the provision of a single-color light beam with components substantially in the cyan spectral range, wherein the individual light beams are combined to form a common light beam by way of the beam combiner, wherein the individual light beams have separate light paths. Apart from the independence of the light source for the provision of single-color light beams with components substantially in the blue spectral range, this embodiment substantially corresponds to the embodiment described in the paragraph above; the explanations given there therefore apply accordingly. In this case, the associated beam combiner must be able to combine four single-color light beams on separate light paths into a common light beam.

By preference, the white-light source according to the invention furthermore comprises a sensor for monitoring the at least one LARP light source (fault sensor, in particular integrity sensor). Since the phosphors used in the conversion element are exposed to high thermal loads, breaks and disturbances in the structure of the phosphor may arise in the event of an overload. The intensity of the emitted light may drop off sharply as a result, and so the splitting-off of appropriate sample light for continuously or repeatedly monitoring the light power is advantageous.

However, what may also arise under certain circumstances is that the laser radiation used to excite the conversion element is inadvertently reflected and directly input coupled into the illumination device. Since blue laser radiation in particular may have a very damaging effect on biological tissue structures and cells, in particular on the retina and the optic nerve in the eye, even at low powers, an immediate shutdown of the illumination device or of the excitation laser, or at least a blocking of the damaging radiation components, is required. This may also be implemented by way of appropriate power monitoring of all light beams and/or by way of an evaluation of the spectral properties of the light beams provided. For example, should a significant increase be detected in the spectral range of an excitation radiation, there may be an automated appropriate reaction.

Hence, the sensor may be designed in particular to monitor the intensity of the energy-rich blue or violet components in the provided spectrum. Should a sudden strong increase in intensity be registered for these components, there can be at least a safety shutdown of the excitation of the corresponding LARP light source or complete down-regulation of the corresponding LARP light source or of the entire white-light source according to the invention. Alternatively, it is also possible to measure and monitor the ratio of the intensity of the provided white light (or of the overall intensity in the event of a single-color or multi-color light that deviates from white light as a result of a corresponding control of the white-light source according to the invention) to the blue spectral component. The sensor may be arranged at a suitable position in the beam path of the white-light source according to the invention.

A further aspect of the present invention relates to an intraocular illumination device, comprising a white-light source according to the invention, an optical fiber for intraocular illumination and a fiber coupling means (optical system) for input coupling the common light beam into a proximal end of the optical fiber.

By preference, the optical fiber has an active diameter less than or equal to 0.1 mm and is capable of emitting a luminous flux of more than 1 lm at its distal end.

The light power required for intraocular illumination devices is between approximately 1 lm and 40 lm. This depends on the respective situation, the tools employed and the specific eye condition of the patient. Using conventional LED light sources, the prior art allows for a maximal introduction of approx. 20 lm into 23G fibers with an active diameter of 0.486 mm and an NA of 0.5. Scaling down linearly, this means that a maximum of only approx. 0.85 lm can be introduced into a corresponding fiber with an active diameter of 0.1 mm if the NA is the same and conventional LED light sources are used. On account of the high étendue of the at least one LARP light source, this value can be increased significantly by way of a white-light source according to the invention. For the preferred active diameters of the fibers between approximately 0.05 mm and 0.1 mm, it is consequently possible to input couple at least 1 lm, preferably between 2 lm and 3 lm, and even more preferably up to 30 lm, into the respective fibers.

Further preferred configurations of the invention arise from the features specified in the respective dependent claims.

The various embodiments of the invention that are set forth in this application can advantageously be combined with one another, unless specifically stated otherwise.

### BRIEF DESCRIPTION OF THE DRAWINGS

The invention will be explained below in exemplary embodiments with reference to the associated drawing, in which:

FIG. 1 shows an exemplary schematic illustration of a fiber-coupled LARP light source according to the prior art,

FIG. 2 shows an exemplary schematic illustration of a first embodiment of a white-light source according to the invention with three independent light sources,

FIG. 3 shows an exemplary schematic illustration of a LARP light source for the provision of two single-color light beams,

FIG. 4 shows an exemplary schematic illustration of a second embodiment of a white-light source according to the invention with four independent light sources,

FIG. 5 shows an intraocular illumination device according to the invention,

FIG. 6 shows a typical spectrum of a white-light LED, and

FIG. 7 shows a typical spectrum of a white-light LARP.

### DETAILED DESCRIPTION OF THE DRAWINGS

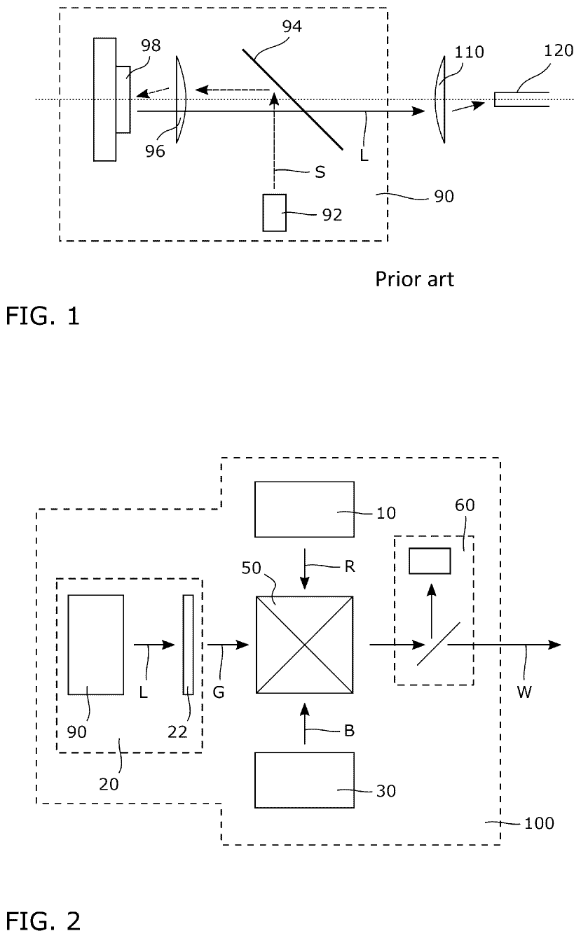

FIG. 1 shows an exemplary schematic illustration of a fiber-coupled LARP light source 90 according to the prior art. In this case, the excitation radiation S emitted by a laser diode 92 (excitation laser) is for example deflected by way of a dichroic mirror 94 and focused on an appropriately adapted conversion element 98 by means of a focusing optics unit 96. Phosphor is used as conversion element 98 in a LARP light source 90. In this case, the phosphor is usually arranged on a substrate or carrier in the form of a heat sink. The conversion light L emitted by the excited conversion element 98 at the focus of the focusing optics unit 96 is subsequently confocally collected and collimated by the focusing optics unit 96. Following the passage through the dichroic mirror 94, the conversion light L can be input coupled into a light guide 120, for example an optical fiber, by way of an appropriate fiber coupling means 110. A corresponding LARP light source 90 may be constructed on an individual basis from individual components or purchased as a fully integrated component part. By way of example, the LARP light source 90 shown only represents one possible variant for constructing such a light source; a multiplicity of further construction variants are sufficiently well known to a person skilled in the art from the prior art.

FIG. 2 shows an exemplary schematic illustration of a first embodiment of a white-light source 100 according to the invention with three independent light sources 10, 20, 30. The white-light source 100 shown for fiber-based intraocular illumination with light of controllable spectral composition comprises one light source 10, 20, 30 each for the provision of single-color light beams R, G, B with respective components substantially in the blue, the green and the red spectral range; and a beam combiner 50 (what is known as an X-cube) for combining the individual light beams R, G, B to form a common light beam W; wherein the white-light source 100 is configured for individual control of the proportions of the individual light beams R, G, B in the common light beam W; wherein one of the light sources 20 is a laser-activated remote phosphor light source, LARP light source, 90 having a phosphor as conversion element (cf. conversion element 98 in FIG. 1) and a laser diode (cf. laser diode 92 in FIG. 1) for exciting the conversion element (see above) by means of an excitation radiation (cf. excitation radiation S in FIG. 1) emitted by the laser diode (see above).

In particular, the shown LARP light source 90 may be designed to emit white light (white-light LARP), and the adaptation of the spectral bandwidth of the emitted white light for the provision of the associated single-color green light beam G may be implemented by way of a spectral filter element 22 between the corresponding LARP light source 90 and the beam combiner 50. The spectral filter element 22 may also take the form of an element of the beam combiner 50, for example a dielectric filter layer applied directly to the surface of the beam combiner 50 or to an internal interface of the beam combiner 50. By preference, the beam combiner 50 mixes spectral components such that no losses occur where possible, and there is a summation of the input powers.

However, according to the invention, the at least one LARP light source 90 may also be designed directly for the provision of the associated colored light beam (the green light beam G in the present case). In particular, there may be a direct provision of the associated colored light beam G by an adapted combination of phosphor and laser diode within the at least one LARP light source 90.

Furthermore, an optional additional sensor 60 for monitoring the at least one LARP light source 90 is depicted in the beam path; for example, said sensor is able to split off a component of the combined light beam W to a detector configured for monitoring the intensity in a specific spectral range. Should a sudden increase in the light intensity be determined, the white-light source 100 according to the invention may be down-regulated or down-controlled accordingly in order to protect the user or the patient (fault sensor, in particular integrity sensor for monitoring the conversion element). In the case of open-loop control, it is possible to set a specific value for the light intensity that should be provided. In the case of closed-loop control, the system behavior may be determined at certain times and may be controlled accordingly. The sensor may be arranged at a suitable position in the beam path of the white-light source according to the invention.

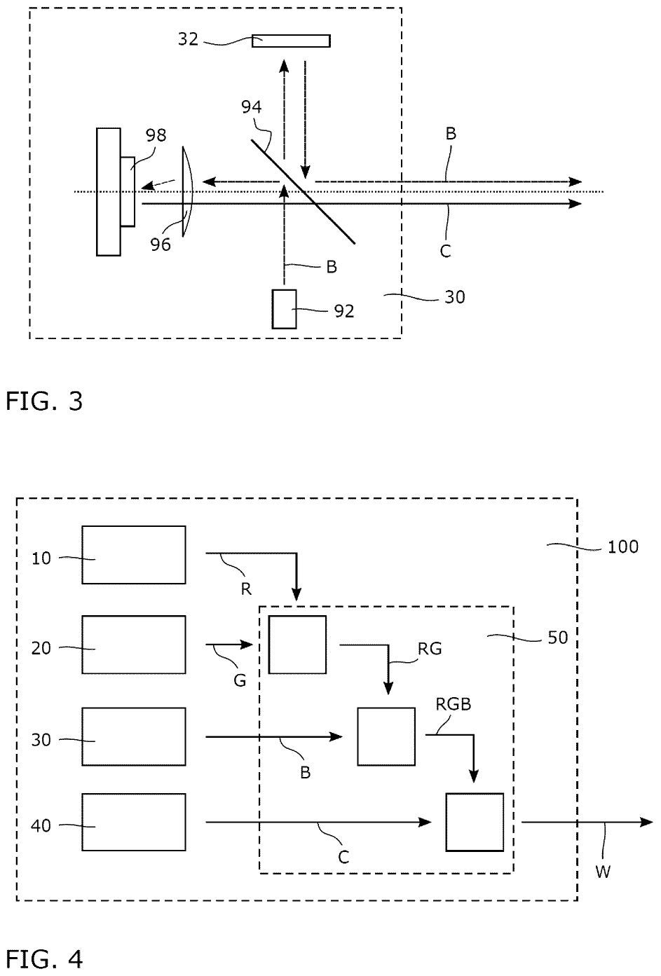

FIG. 3 shows an exemplary schematic illustration of a LARP light source for the provision of two single-color light beams. The basic structure of the LARP light source 90 corresponds to the embodiment shown in FIG. 1; the individual reference signs and their assignment therefore apply accordingly. In the illustration, provision of the colored light beam B with components substantially in the blue spectral range is implemented by way of a laser diode 92 emitting light substantially in the blue spectral range. By way of the laser diode 92 there is an additional excitation of a conversion element 98 for the provision of a single-color light beam C with components substantially in the cyan spectral range. In this case, the dichroic mirror 94 may be designed as a beam splitter such that a component of the blue excitation light B can be reflected off a reflector 32 disposed downstream thereof and overlaid at the dichroic beam splitter 98 on the created conversion light from the single-color light beam C with components substantially in the cyan spectral range. For example, such a light source could be used in the embodiment of a white-light source 100 shown in FIG. 2 as the third light source 30 for the provision of two single-color light beams (B, C) with respective components substantially in the blue spectral range B and in the cyan spectral range C.

In an alternative embodiment of a LARP light source for the provision of two single-color light beams (e.g. in the colors of cyan and blue), a particularly compact uniaxial beam profile without an additional beam splitter may be realized by the use of a transparent conversion element (e.g. a cyan phosphor). For example, the light of a blue LARP light source (or a white-light LARP with a high blue component) can be used directly for the excitation of a corresponding transparent conversion element. By way of the excitation, some of the transmitted blue light is then converted into cyan-colored light, and so there is a superposition of blue and cyan-colored components downstream of the conversion element.

FIG. 4 shows an exemplary schematic illustration of a second embodiment of a white-light source 100 according to the invention with four independent light sources. In addition to the embodiment shown in FIG. 2, the white-light source 100 furthermore comprises a LARP light source (for example according to FIG. 1) as light source 40 for the provision of a single-color light beam C with components substantially in the cyan spectral range, wherein the individual light beams R, G, B, C are combined to form a common light beam W by way of the beam combiner 50, and wherein the individual light beams R, G, B, C have separate light paths. By way of example, the beam combiner 50 shown is a sequence of individual beam combiners, in which two respective input beams on separate light paths are combined into a common light path.

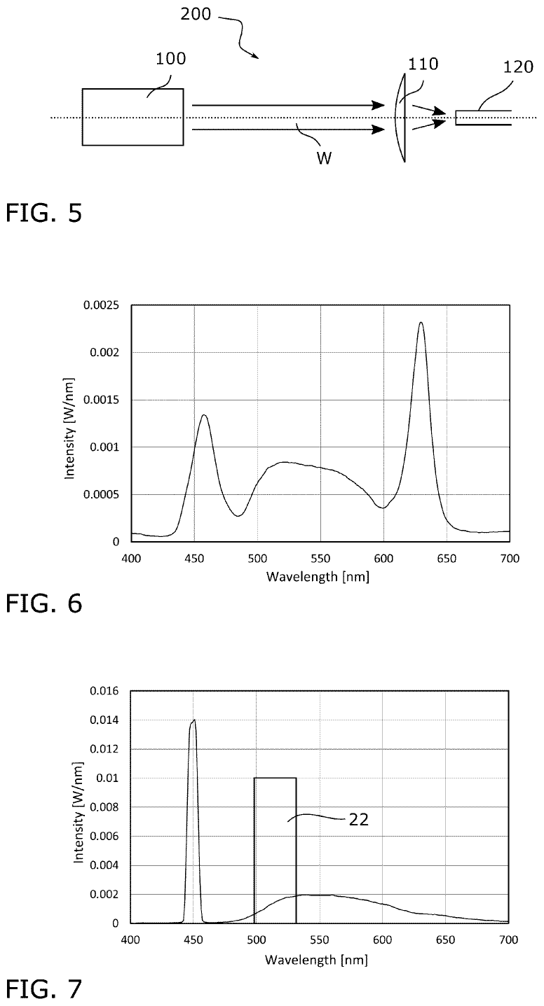

FIG. 5 shows an intraocular illumination device 200 according to the invention. The intraocular illumination device 200 shown comprises a white-light source 100 according to the invention; a light guide 120 for intraocular illumination and a fiber coupling means 110 (optical system) for input coupling the common light beam W into a proximal end of the light guide 120. By preference, the light guide 120 takes the form of an optical fiber, wherein the optical fiber has an active diameter of less than 0.1 mm and is capable of emitting a luminous flux of more than 1 lm at its distal end.

FIG. 6 shows a typical spectrum of a white-light LED. To this end, for example, a respective red, green and blue light-emitting diode may be combined with one another in a common LED housing to form what is known as an RGB-LED, with the result that a spectrum that appears to be white is emitted to the outside. For improved color rendering with a broader spectral scope, a component of the light from what is known as a primary LED may also be shifted into another spectral range by way of a conversion element. Depending on the phosphor, such conversion spectra may extend over a spectral width of up to several 100 nm. In this case, high-energy blue light or light in the ultraviolet range from an appropriate LED is usually used as excitation light for the conversion.

Clear peaks at approximately 460 nm (blue) and approximately 630 nm (red) may be identified in the spectrum shown. By contrast, the greenish yellow spectral range between approximately 597 nm and approximately 580 nm exhibits the typical flat profile of a conversion spectrum from a phosphor. The resultant white-light spectrum is strongly modulated and has clearly identifiable drops in intensity, especially at the individual range transitions (at approximately 480 nm and approximately 600 nm). It is possible to obtain a more uniform spectrum by attenuating the individual peaks; however, the overall intensity available for emission is significantly reduced in the process.

By contrast, FIG. 7 shows a typical spectrum of a commercial white-light LARP. In this case, a blue excitation laser emitting at 450 nm was used to excite the conversion element. The excitation spectrum is substantially narrower than in the case of LEDs (the spectral width shown is limited by the utilized spectrometer). A broad conversion spectrum between approximately 470 nm and approximately 700 nm can be created by an effective excitation of the conversion element. The spectrum has a flat curve very similar to the solar spectrum, without an identifiable modulation. On account of its broader scope of color, such a spectral curve allows significantly improved color rendering vis-à-vis the light from white-light LEDs or RGB LEDs. Moreover, the spectrally non-overlapping excitation light may be filtered out by way of a suitable low-pass filter without influencing the conversion spectrum.

Additionally, the drawing plots by way of example the filter function of a bandpass filter operating in the green spectral range between approximately 497 nm and approximately 530 nm, as may be used in a white-light source 100 according to the invention (cf. FIG. 2) as spectral filter element 22 in a light source 20 for the provision of a single-color light beam G with components substantially in the green spectral range. However, use could also be made of bandpass filters with substantially broader filter bandwidths. For example, for an extended green spectral range, it is possible to provide a spectral filter element with a pass range between approximately 497 nm (green) and approximately 575 nm (greenish yellow).

### LIST OF REFERENCE SIGNS

10 First light source (red)
20 Second light source (green)
22 Spectral filter element
30 Third light source (blue)
32 Reflector
40 Fourth light source (cyan)
50 Beam combiner
60 Sensor (e.g. fault or integrity sensor)
90 LARP light source
92 Laser diode (LD)
94 Dichroic mirror
96 Focusing optics unit
98 Conversion element (e.g. phosphor(s))
100 White-light source
110 Fiber coupling means
120 Light guide
200 Intraocular illumination device
S Excitation radiation
L Conversion light (e.g. single color, white or hyperspectral)
R First light beam (red)
G Second light beam (green)
B Third light beam (blue)
C Fourth light beam (cyan)
W Combined light beam (white)

1. A white-light source for fiber-based intraocular illumination with light of controllable spectral composition, comprising:
at least two light sources for the provision of light beams of different colors, preferably with components substantially in the blue, the green and the red spectral range, with the red spectral range extending between wavelengths of approximately 640 nm and approximately 780 nm, the green spectral range extending between wavelengths of approximately 490 nm and approximately 570 nm and the blue spectral range extending between wavelengths of approximately 430 nm and approximately 490 nm;
wherein the individual light beams are combined to form a common light beam (W);
wherein the white-light source is configured for individual control of the proportions of the individual light beams in the common light beam (W);
wherein at least one of the light sources is a laser-activated remote phosphor light source, LARP light source, having a phosphor as conversion element and a laser diode for exciting the conversion element by means of an excitation radiation(S) emitted by the laser diode.

2. The white-light source as claimed in claim 1, wherein the white-light source comprises one light source each for the provision of single-color light beams (R, G, B) with respective components substantially in the blue, the green and the red spectral range.

3. The white-light source as claimed in claim 1, wherein the white-light source comprises a beam combiner for combining individual light beams to form the common light beam (W).

4. The white-light source as claimed in claim 1, wherein the conversion element comprises an auxiliary phosphor for adapting the conversion element to the excitation radiation(S) of the laser diode and/or the conversion element comprises a plurality of phosphors for specifying a converted color spectrum.

5. The white-light source as claimed in claim 1, wherein the at least one LARP light source is designed to emit white light.

6. The white-light source as claimed in claim 5, wherein a spectral curve of the emitted white light is adapted for the provision of the associated light beam by way of a spectral filter element between the LARP light source designed to emit white light and the beam combiner or by way of a spectral filter element as an element of the beam combiner.

7. The white-light source as claimed in claim 1, wherein the at least one LARP light source is directly designed for the provision of the colored light beam (R, G, B).

8. The white-light source as claimed in claim 7, wherein a direct provision of the colored light beam (R, G, B) is implemented by an adapted combination of phosphor and laser diode within the at least one LARP light source

9. The white-light source as claimed in claim 2, wherein the provision of the colored light beam (B) with components substantially in the blue spectral range is implemented by way of a diode or laser diode emitting light substantially in the blue or violet spectral range, and the provision of the colored light beams (R, G) with components substantially in the red and green spectral ranges is implemented by means of one LARP light source each, the violet spectral range extending between wavelengths of approximately 380 nm and below approximately 430 nm and the blue spectral range comprising a cyan spectral range at least in part, the latter extending between wavelengths of approximately 482 nm and approximately 494 nm.

10. The white-light source as claimed in claim 2, wherein the provision of the colored light beam (B) with components substantially in the blue or violet spectral range is implemented by way of a laser diode emitting light substantially in the blue or violet spectral range, and the laser diode additionally brings about an excitation of a conversion element for the provision of a single-color light beam (C) with components substantially in the cyan spectral range, the violet spectral range extending between wavelengths of approximately 380 nm and below approximately 400 nm and the cyan spectral range extending between wavelengths of approximately 482 nm and 494 nm.

11. The white-light source as claimed in claim 2, furthermore comprising a LARP light source as light source for the provision of a single-color light beam (C) with components substantially in the cyan spectral range, wherein the individual light beams (R, G, B, C) are combined to form a common light beam (W) by way of the beam combiner wherein the individual light beams (R, G, B, C) have separate light paths, and wherein the cyan spectral range extends between wavelengths of approximately 482 nm and approximately 494 nm.

12. An intraocular illumination device comprising:
a white-light source as claimed in claim 1;
a light guide for intraocular illumination; and
a light guide coupling means for input coupling the common light beam (W) into a proximal end of the light guide

13. The intraocular illumination device as claimed in claim 12, wherein the light guide takes the form of an optical fiber, wherein the optical fiber has an active diameter of less than 0.1 mm and is capable of emitting a luminous flux of more than 1 lm at its distal end.
